(19) United States
(12) Patent Application Publication
(10) Pub. No.: US 2023/0022060 A1
(43) Pub. Date: Jan. 26, 2023

(54) TURKESTERONE HYDROXYPROPYL B-CYCLIC DEXTRIN COMPLEX AND METHOD OF MANUFACTURING THEREOF

(71) Applicant: Gorilla Mind, LLC, Eagle, ID (US)

(72) Inventor: Christopher Deoudes, Meridian, ID (US)

(21) Appl. No.: 17/812,608

(22) Filed: Jul. 14, 2022

Related U.S. Application Data
(60) Provisional application No. 63/221,692, filed on Jul. 14, 2021.

(51) Int. Cl. A61K 47/69; A61K 36/53; A61K 47/61; A23L 33/125; A23L 33/105; A23L 3/50; A23L 3/00

(52) U.S. Cl. CPC A61K 47/6951; A61K 36/53; A61K 47/61; A23L 33/125; A23L 33/105; A23L 3/50; A23L 3/001; A61K 2236/39; A61K 2236/53; A61K 2236/51; A23V 2002/00

(57) ABSTRACT

The present invention provides an advanced absorption and delivery system to an ecdysterone-based nutritional supplement, specifically a turkesterone-based nutritional supplement, a turkesterone hydroxypropyl β-cyclic dextrin complex manufactured using a co-precipitation technique, the manufacturing process including a fluid bed drying system wherein the complex is dried using hot air blown into contact with a fluidized bed.

### FIELD OF THE INVENTION

The present invention relates to nutritional supplements and the manufacture thereof. More specifically, the present invention relates to a Turkesterone-based nutritional supplement and methods of manufacturing the same.

### BACKGROUND OF THE INVENTION

Cyclodextrin

Cyclodextrins (CD) (or cycloamyloses, cyclomaltoses and Schardinger dextrins) are cyclic oligosaccharides consisting of glucopyranosyl units linked by α-(1,4) bonds. The widely used natural cyclodextrins are α-, β- and γ-cyclodextrin consisting of 6, 7 and 8 glucopyranose units, respectively. The main properties of those cyclodextrins are given in Table 1. The cyclodextrin molecules have a unique structure with a hydrophobic cavity and a hydrophilic surface in which a guest molecule can be entrapped. Cyclodextrin can form inclusion complex with a wide variety of solid, liquid and gaseous compounds. Complex formation is a dimensional, geometrically limited fit, between cyclodextrin cavity and the guest molecule.

Generally, hydrophobic molecules have greater affinity for the cyclodextrin cavity when they are in water solution. Moreover, the encapsulation changes the physical and chemical properties of the guests, such as solubility and stability. Therefore, cyclodextrins are suitable for application in food and flavors, pharmaceutical products, cosmetic, agriculture and chemcical industries. Cyclodextrins can link specifically to other cyclodextrins (covalent or noncovalent). Because of this property, cyclodextrins can be used as building blocks for the construction of supramolecular complexes. Building blocks which cannot be prepared by common methods can be applied, such as separation of complex mixtures of molecules and enantiomers. Cyclodextrins are produced by reacting gelatinized starch with the enzyme cyclodextrin glucosyltransferase (CGTase) as a result of intramolecular transglycosylation reaction from degradation of starch.

Inclusion Complex Formation

Cyclodextrins have an internal non-polar hole and hydroxyl groups placed on the surface, the inclusion of hydrophobic compounds takes place mainly by hydrophobic interactions between guest molecules and the walls of cyclodextrin cavity. However, other forces, such as van der Walls and dipole—dipole interactions, may be involved in the binding of the guest. Despite the number of factors and different forces involved in the complexation with cyclodextrins, the production of complexes is a rather simple process. There are several methods to obtain cyclodextrin—guest complexes depending on the properties of the guest and the nature of the chosen cyclodextrin.

Kneading Method

Kneading technique is suitable for poorly water-soluble guests, because the guest is dissolved slowly during the formation of complex. It affords a very good yield of inclusion formation but it is unsuitable for large scale preparation. Firstly, the liquid or dissolved solid guest is added to slurry of cyclodextrin and kneaded (in a mortar), and then the paste is dried. The obtained solid is washed with a small amount of solvent to remove the free particles adsorbed on the cyclodextrin surface and then dried under vacuum. The inclusion complex formation of cyclodextrins by kneading method has been reported in the encapsulation of ibuprofen, omega-3 fatty acids in thymol essential oil, thyme essential oil and European anchovy (Engraulis encrasicolus L.) oil.

Co-Precipitation Method

Co-precipitation technique is useful for non-water-soluble substances. Poor yields are obtained from this method because of the competitive inhibition from organic solvents used as the precipitant. The guest is dissolved in organic solvents (such as chloroform, benzene and diethyl ether, etc), and appropriate amount of cyclodextrin dissolved in water is added with agitation. The solution is cooled and complex crystals occur. The crystals are washed with organic solvent and then dried at 50° C. The co-precipitation technique has previously applied for encapsulation of drugs such as oxaprozin and trans-anethole (major component of anise and fennel essential oils).

Co-Precipitation Method Based on Phase Solubility

In the co-precipitation technique, the solid inclusion complex could be recovered from the saturated aqueous solution. This technique is not for a system which has an A-type phase solubility diagram. And it is not suitable for large-scale preparations because of large quantities of water and time consuming. The amounts of host and guest used are estimated from the BS-type phase-solubility diagrams (no more undissolved guest and cyclodextrin are still within its solubility limit). Cyclodextrin and guest are dissolved in hot water and cooled slowly. The precipitate inclusion powder is separated by filtration and it is then dried.

Heating in a Sealed Container

After adsorbing a definite amount of water vapor, a physical mixture of active compound and the host molecule is sealed in a container and heated to a temperature ranging from 43° C. to 142° C. to obtain a crystalline inclusion compound. This technique is also performed under nitrogen gas pressure and can be used for thermostable volatiles.

Freeze-Drying or Lyophilization

The freeze-drying technique is suitable for thermolabile or water soluble guests. The required proportion of cyclodextrin and the guest molecule are dissolved in water with stirring. The solution is freeze-dried and the obtained powder is washed with organic solvent and then dried under vacuum. This method can produce a very good yield of inclusion complex and it is possible to scale up. Comparing with other available techniques, freeze-drying technique has been wildly applied for cyclodextrin inclusion complex formation, especially water soluble hydroxyproply-β-cyclodextrin. Several essential oils and their pure major active compounds have been encapsulated in hydroxyproply-β-cyclodextrin. These include cinnamon and clove, estragole (major component of basil and tarragon essential oils), black pepper essential oil, thymol and thyme essential oil, kamebakaurin (kaurane diterpene), ITH12674 (multitarget drug) and chloramphenicol.

Spray Drying

Cyclodextrin and guest molecule are dissolved in deionized water and then the solution is dried by the spray-dryer. The spraydryer is operated under the most appropriate conditions such as inlet temperature and sample feeding speed; As temperatures of 50-70° C. are used, this technique is only used for thermostable molecules. Recently, the spray-drying technique has been used for encapsulation of folic acid in cyclodextrin.

Inclusion Complex Confirmation

Inclusion complex formation can be confirmed by studying the interaction between a guest molecule and cyclodextrin using various techniques.

Phase Solubility

The persistence of an inclusion complex in aqueous solution does not guarantee the existence of the same complex in the crystalline state. Therefore, the powder derived from inclusion complexation must be determined whether it is an inclusion complex or just a physical mixture of the guest and cyclodextrin molecules. The limited water soluble organic compounds frequently increase their water solubility in the presence of cyclodextrins because of the formation of water soluble complexes between the dissolved cyclodextrin and the guest molecule. The stability of the complex form is characterized by the stability (or equilibrium) constant, Ks, of the complex.

$${K}_{s} = \frac{kr}{kd} = \frac{[ complex ]}{[ B - CO ] [ guest ]}$$

where kr (M-1 s-1) is the recombination rate constant and kd (s-1) is the dissociation rate constant.

The greater Ks value the greater stability of the complex. In solution, the fundamental parameters for inclusion compound formation (such as stability constant, stoichiometry and thermodynamic parameters) can be accurately obtained and the equilibrium of complexes and free compounds can be managed by altering the environmental conditions (such as concentration, temperature, pH and polarity of the solvent), by addition of a competitive molecule, or by choosing the most suitable cyclodextrin or its derivative.

Higuchi and Connors have established a classification of the complexes from the phase solubility profiles derived from the interaction between the guest and the host in the solution. A-type curves suggest the formation of soluble inclusion complexes. B-type indicates the formation of inclusion complexes with poor solubility. BS-type reveals complexes of limited solubility and a BI-type curve shows the formation of insoluble complexes. A-type curves are subdivided into AL-type (linear enhances of guest solubility as a function of cyclodextrin concentration), AP-type (positively deviating isotherms) and AN-type (negatively deviating isotherms) subtypes.

Ks can be obtained from the linear portion of the phase solubility diagrams by the Eq.2:

$${K}_{s} = \frac{[ slope ]}{[ intercept ] [ 1 - slope ]}$$

where intercept is the dissolved guest in the aqueous complexation medium when no ligand (cyclodextrin) is present.

Complexes with stability constants (Ks) about 100-5000 L/mol, seem to be suitable for practical applications. Weak interaction of the very labile complexes (Ks<100) results in premature release of the guest and insignificant improvement in solubility. In cases of very high Ks (Ks>5000), the complexes are very stable and the release of the guest from the cyclodextrin cavity is incomplete or obstructed. This property can be applied for modification of drug or fragrances release especially slow release control.

In some cases of small Ks values, complexation promotes finer physicochemical, biopharmaceutical and pharmaco-technical properties of drugs or other guest molecules. For inclusion, complex formation in solution, the molar ratio of host to guest molecules is usually 1:1, except for complex formation with long-chain or bifunctional guest molecules (e.g. have two aromatic rings on opposite sides of a small central molecule segment).

As most flavor components are monoterpenoids and sesquiterpenoids and phenylpropane derivatives of an average molecular weight of 120-160, a 1:1 complex formation is observed. But there are reports of complexes exhibiting other host: guest molar ratios, such as β-cyclodextrin: allyl isothiocyanate (1:2) and β-cyclodextrin:(β)-α-bisabolol (2:1).

Differential Scanning Calorimetry (DSC)

The inclusion complex can be confirmed using DSC indirectly by comparing the thermal stability of the free compound with the encapsulated form. At the temperature of its melting point or boiling point, an endothermic peak can be observed for the compounds and the physical mixture but will be absent for the complex used DSC technique to characterize the formation of inclusion complexes of β-cyclodextrin with essential oils from cinnamon and clove. The exothermic peaks at approximately 265° C. and 260° C. could be interpreted as resulting from the hydrolysis or oxidation of trans cinnamaldehyde and eugenol of cinnamon and clove oil, respectively. The peaks of inclusion complex of the tested essential oils with β-cyclodextrin were not detected in the thermogram, indicating active compounds were protected within the cavity of the β-cyclodextrin. The exothermic peaks around 300° C. for the β-cyclodextrin sample due to melting and thermal decomposition of the β-cyclodextrin itself. Besides UV-vis spectra, Phase solubility study and Differential Scanning calorimetry (DSC), there are other several techniques to characterize inclusion complex formation of cyclodextrin which are Infrared Spectroscopy, Vacuum Methods, X-ray Diffraction, Chromatography, Mass Spectrometry, Nuclear Magnetic Resonance Spectroscopy, Fluorescence Spectroscopy and Optical Methods.

Cyclodextrin Derivatives

Although β-cyclodextrin can be used in several fields and is fitted to many types of guest, its solubility in water is quite low. About 14 g α-cyclodextrin or 23 g γ-cyclodextrin can be dissolved in 100 mL water at 25° C., while only 1.8 g of β-cyclodextrin can be dissolved. β-cyclodextrin has a low solubility in water because of intra-molecular and inter-molecular interaction via their hydroxyl group. To improve water solubility, the structure of β-cyclodextrin has been modified. The OH groups on C2, C3 and C6 are points for structural modification. The hydroxyl groups on C6 are the most reactive while the OH groups at C3 are much less reactive than those at C2. By various molecular manipulations, cyclodextrins can be modified to derivatives with different physicochemical properties.

Cyclodextrin derivatives have been developed to extend the physiochemical properties and the inclusion capacity of original cyclodextrins. There are several kinds of cyclodextrin derivatives such as hydrophilic, hydrophobic and ionic derivatives. The hydrophilic cyclodextrins, such as methyl-β-cyclodextrin, 2,6-di-O-methyl-β-cyclodextrin and 2,3,6-per-O-methyl-β-cyclodextrin, alter the release rate of poorly water-soluble drugs, which can be used to enhance drug absorption across biological barriers. On the other hand, the hydrophobic cyclodextrins are used as sustained-release carriers for water-soluble drugs such as protein and peptide drugs. The amorphous hydrophilic cyclodextrins, such as hydroxyalkylated-β-cyclodextrin or hydroxypropyl-β-cyclodextrin, are useful for inhibiting polymorphic transitions and crystallization rates of poorly watersoluble drugs during storage, which can consequently maintain higher dissolution characteristics and oral bioavailability of the drugs. A hydroxyalkylated β-CD derivative is relatively high aqueous solubility with low toxicity and satisfactory inclusion ability. This modified β-cyclodextrin has higher water solubility (above 60 g β-cyclodextrin in 100 mL water) and a proven safe profile.

Applications of Cyclodextrins and Cyclodextrin Derivatives

Due to each guest molecule is individually surrrounded by a cyclodextrin, the molecule is micro-encapsulated from a microscopical point of view leading to advantageous alter in the chemical and physical properties of the guest molecules. Encapsulation in cyclodextrins provides an intimate effect on the physicochemical properties of guest molecules as they are tempolarily locked within the host cavity giving rise to benificial modifications of guest molecules, which are not achievable otherwise. The advantages of these characteristics are solubility improvement of highly insoluble guests, stabilization of labile guests against the degradative effects of environment (oxidation, light and heat), control of volatility and sublimation, physical isolation of incompatible compounds (via chromatography), taste modification by masking off flavours, odour elimination and controlling of drug and flavour release. Therefore, cyclodextrins can be used in several fields as follows:

Application in Foods and Flavors

Cyclodextrins are used in food formulations for flavor protection throughout many rigorous food-processing methods of freezing, thawing and microwaving, and used for flavor preservation to a greater extent and longer period. They also have been used for removal of cholesterol from animal products such as eggs and dairy products, removal of bitter components from citrus fruit juices, removal of phenolic compounds which cause enzymatic browning and enhancement of flavor in alcoholic beverages such as whisky and beer. Aqueous solubility and bitter taste of flavonoids and terpenoids, the plant components which are rich of antioxidant and antimicrobial properties, can be improved by cyclodextrin complexation.

Application in Cosmetics Personal Care and Toiletry

Cyclodextrins can be used for control release of fragrances from the inclusion compounds in perfumes, room fresheners or detergents and used for odor control in diapers, menstrual products, paper towels and washed items. They also used in silica-based toothpastes to increase the availability of triclosan, an antimicrobial agent. Cyclodextrins and their derivatives (such as hydroxypropyl β-CD) are used in sunscreen lotions to reduce the side effects of the formulation by limiting the interaction between the UV filter and the skin, and improve performance and shelf-life of self-tanning emulsions or creams.

Application in Environment Protection

Highly toxic substances can be removed from industrial effluent by inclusion complex formation. In the mother liquor of the insecticide trichlorfon, the uncrystallisable trichlorfon can be converted into a β-cyclodextrin complex and in a single treatment 90% of the toxic material is removed. have pointed out that inclusion of a guest compound by cyclodextrin can prevent from hydrolytic degradation. The effect of inclusion complex formation on antimicrobial activity and antioxidant property of chlorogenic acid and its complex was also tested by. No significant difference of antimicrobial activity against three bacteria; Staphylococcus aureus, Bacillus subtilis and Escherichia coli, was observed between chlorogenic acid and its complex. The 1, 1-diphenyl-2-picrylhydrazyl (DPPH) radical scavenging activities of chlorogenic acid and its complex were not significant (P>0.05). Both exhibited stronger DPPH radical scavenging activity than butylated hydroxytoluene (BHT) (positive control) when a concentration higher than 10 μg/ml was taken.

Antimicrobial activity of allyl isothiocyanate entrapped in α- and β-cyclodextrin was also evaluated by Allyl isothiocyanate entrapped in β-cyclodextrin exhibited the most antimicrobial effect, with minimum initial concentrations with fungistatic effect of 0.5-1 μL/L air and bacteriostatic concentrations of 25 and 50 mL/L for E. coli and L. monocytogenes, respectively. Allyl isothiocyanate entrapped in α-cyclodextrin also effectively inhibited P. expansum with a minimum initial concentration with fungistatic effect of 1 μUL, while allyl isothiocyanate alone inhibited growth at 5 μUL.

Application in Pharmaceuticals

The addition of α- or β-cyclodextrin increases the water solubility of several poorly water-soluble substances to improve bioavailability and increase the pharmacological effect allowing a reduction in the dose of the drug administered. Cyclodextrins also have been used successfully in aqueous dermal formulations, nasal drug delivery systems and several eyedrop solutions. Furthermore, they can be applied to reduce the effects of bitter or irritant tasting and bad smelling drugs. Cyclodextrins have been used for controlled release of drugs such as ciprofloxacin, triclosan, vancomycin and chlorhexidine digluconate. The low water soluble anticancer drug candidates, Pt(IV)-bis(benzoato), was also encapsulated in β-cyclodextrin to enhance water solubility. Cyclodextrin have been applied for encapsulation of antimicrobial drugs such as chloramphenicol. The inclusion complex formation with multicomponent of β-cyclodextrin and amino acid, glycine or cysteine, improved aqueous solubility and maintained microbiological activity of chloramphenicol and reduced the generation of reactive oxygen species (ROS) in leucocytes induced by this drug. Physicochemical and biological properties of chloramphenicol were improved by multicomponent complexation with β-cyclodextrin and N-acetylcysteine preparing by freeze-drying or physical mixture methods. The system was effective to reduce toxicity of chloramphenicol against leukocytes while enhancing its solubility and antibiofilm activity.

The encapsulations of cyclodextrin-drug inclusion complexes in liposome, phospholipid vesicles composed of lipid bilayers enclosing one or more aqueous compartments, have recently been reviewed by. Because of its ability to enhance aqueous solubility of hydrophobic drugs, cyclodextrin can be used to increase drug entrapment in the aqueous compartment of liposomes and liposomes can protect CD/drug inclusion complexes until drug release. Anethole (ANE) (transanethole), a major component of anise and fennel essential oils, was used as a model of a volatile and highly hydrophobic drug.

Application in Entrapment of Essential Oils

Essential oils are natural plant products composing of mixtures of several compounds. Because of their active compounds, they have been used for several fields since ancient time. The components of essential oils can be classified into two groups: (1) hydrocarbons including mono-, di-, and sesquiterpenes and (2) oxygenated compounds including alcohols, aldehydes, esters, ketones, phenols, etc. Besides, they also contain some phytochemicals which play efficient role in biological activities such as flavonoids, terpenoids, carotenoids, coumarins, curcumines, etc. Entrapment of essential oils with β-cyclodextrin has been applied to protect essential oils against the damaging effects of the environment such as oxidation, degradation from heat and light, evaporation, and moisture. Several types of essential oils have been entrapped in β-cyclodextrin.

Other Applications

Cyclodextrins were found to form inclusion complex with several agricultural chemicals including herbicides, insecticides, fungicides, repellents, pheromones and growth regulators. They can also be applied to delay germination of seed. Cyclodextrins have also been applied encapsulation of phenolic compounds. The encapsulation of rosmarinic acid, an efficient phenolic antioxidant from rosemary with a marketing authorization, with β-cyclodextrin was also characterized by using 1H NMR (1D- and 2D-ROESY). They also found that rosmarinic acid spontaneously formed a relatively stable inclusion complex with CD in water.

Recently, cyclodextrins were applied in the encapsulation of lipase for biodiesel production. For enzymatic biodiesel production, the methanol tolerant of lipase, a biocatalyst, is a critical parameter. The methanol resistance of Yarrowia lipolytica Lipase 2 (YLLIP2) was significantly improved after encapsulated in β-cyclodextrin which exhibited approximately 7000 U/mg specific activity in 30 wt % methanol for 60 min compared with no activity of the free enzyme under the same conditions. β-cyclodextrin molecules weakened the conformational change of the enzyme and maintained a semi-open state of the lid by overcoming the interference caused by methanol molecules.

Nevertheless, there remains a dearth of information on the application of such manufacturing and delivery methods in the field of nutritional supplements. Indeed, there appears to be little scholarship on the use of such methods in Turkesterone or other Ecdysterone delivery through nutritional supplements or on the manufacturing processes of such nutritional supplements. There thus remains a need in the supplements industry for a product utilizing such manufacturing and delivery methods on Turkesterone- and other Ecdysterone-based nutritional supplement products.

### SUMMARY OF THE INVENTION

It is thus an object of the present invention to provide an advanced absorption and delivery system to a Ecdysterone-based nutritional supplement. It is a further object of the present invention to provide an advanced absorption and delivery system to a Turkesterone-based nutritional supplement. It is a further object of the present invention to provide a method of manufacturing nutritional supplements employing such advanced absorption and delivery systems.

To accomplish these and other objectives, the present invention provides a turkesterone hydroxypropyl β-cyclic dextrin complex manufactured using a co-precipitation technique. This technique is useful for non-water-soluble substance. Poor yields are often obtained from this method because of the competitive inhibition from organic solvents (such as chloroform, benzene and diethyl ether, etc), and appropriate amount of cyclodextrin dissolved in water is thus added with agitation. The solution is cooled and complex crystals occur. The crystals are washed with organic solvent and then dried at 50° C. The present invention further provides a more generic ecdysterone hydroxypropyl β-cyclic dextrin complex manufactured using the same co-precipitation technique.

Preferable embodiments of the complex employ ajuga turkestanica extract standardized to 10% turkesterone complexed with the hydroxypropyl-beta-cyclodextrin complex, with a preferable delivery amount of 500 milligrams. Even more preferably, the complex employs a one-to-one formulation of ajuga turkestanica extract with hydroxypropyl-beta-cyclodextrin, with preferable amounts of 250 milligrams of each per dose. The complex is preferably delivered orally into the user's system, and preferably takes the form of a capsule or pill, although other forms, such as a powder, are possible as well.

The goal of the fluid bed drying and complexing manufacturing process is to achieve the one-to-one ratio described above. To begin, cyclodextrin is preferably added to a mixing tank along with hot water, Ajuga, and an organic, food grade thickening excipient. The ingredients are mixed in the mixing tank for a predetermined period of time and then slowly cooled down to cause precipitation. The precipitant contains the Ajuga embedded in cyclodextrin together with the food grade thickening excipient.

The precipitant is next separated using a filtration system and inserted into a storage tank, after which the precipitant is dried using hot air blown into direct or indirect contact with a fluidized bed. The hot air is preferably in the range of 50-70 degrees Celsius. Samples of the dried precipitant are tested to verify potency and moisture levels. In preferable embodiments, the dried precipitant should have a moisture content between 3% and 5%.

Once the potency and moisture content have been verified, the dried precipitant is milled and filtered to ensure proper sizing of the granules, preferably using a 16-20 mesh. The granulized, dried precipitant is then introduced to a rotary mixer and then packaged as a finished product. Samples are preferably taken throughout the manufacturing process to test for quality and effectiveness of process.

The preceding summary of the present invention is not limiting, as alternative embodiments, arrangements, and methods will be recognizable to those of skill in the art. Other objects of the present invention and its particular features and advantages will become more apparent from consideration of the following drawings and the detailed description of the present invention provided herein.

### BRIEF DESCRIPTION OF THE DRAWINGS

FIG. 1 depicts a schematic representation of a manufacturing process for a turkesterone hydroxypropyl β-cyclic dextrin complex according to exemplary embodiments of the present invention.

FIG. 2 depicts a schematic representation of a fluid bed drying system according the exemplary embodiments of the present invention depicted in FIG. 1.

### DETAILED DESCRIPTION OF THE INVENTION

The following detailed description illustrates the technology by way of example, not by way of limitation of the principles of the invention. This description will enable one skilled in the art to make and use the invention, and describes several embodiments, adaptations, variations, alternatives, and applications of the invention, including what is presently believed to be the best mode of carrying out the invention. The present invention is not limited to that described hereafter, however, and those of skill in the art will recognize other manners of carrying out the invention without departing from the principles thereof.

Described herein are preferable embodiments of nutritional supplements employing an advanced absorption and delivery system. The present invention applies this advanced absorption and delivery system specifically to Ecdysterones, and more specifically to Turkesterone. Preferable embodiments of the present invention thus provide a turkesterone hydroxypropyl β-cyclic dextrin complex manufactured using a co-precipitation technique. The Turkesterone is preferably derived from ajuga turkestanica extract standardized to 10% turkesterone and then complexed with the hydroxypropyl-beta-cyclodextrin complex. A per-dose delivery of 500 milligrams is preferable.

The turkesterone hydroxypropyl β-cyclic dextrin complex preferably employs a one-to-one formulation of ajuga turkestanica extract with hydroxypropyl-beta-cyclodextrin, with preferable amounts of 250 milligrams of each per dose. The complex is preferably delivered orally into the user's system, and preferably takes the form of a capsule or pill. Other user intake methods may also include providing a powder that a user mixes with a liquid, as is well known in the nutritional supplement industry.

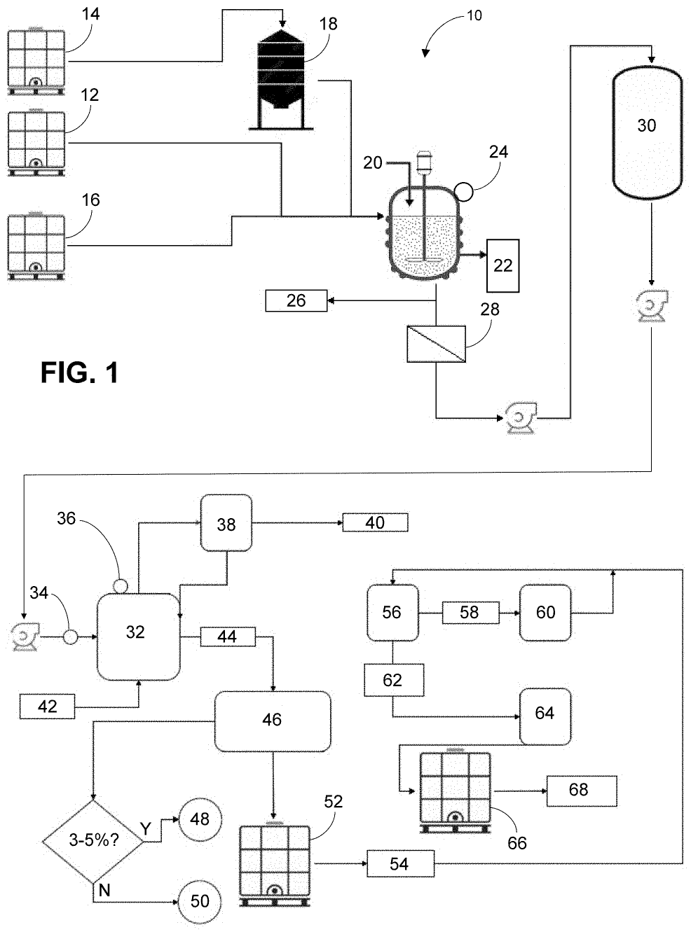

To achieve the one-to-one ratio complexing of the ajuga turkestanica extract with the hydroxypropyl-beta-cyclodextrin complex, the preferable manufacturing process 10 depicted in FIG. 1 is employed in preferable embodiments of the present invention. The process 10 begins by combining the ajuga turkestanica extract 12, cyclodextrin 14, and an organic, food grade thickening excipient 16 with hot water in a mixer 20 for heating and combination. The organic, food grade thickening excipient 16 may be any known agent, such as xanthem gum or the like, as will be known by persons of skill in the art. The cyclodextrin may be held in a storage unit 18, as depicted, before being introduced into the mixer 20.

The mixer 20 heats and agitates the mixture for a period of time sufficient to ensure proper combination. Preferable embodiments of the mixer 20 are equipped with a reaction progress monitoring system 22, which obtains data about the mixture's status, and at least a temperature indicator 24, to determine when the mixture is sufficiently heated and processed to ensure proper combination. The mixture is then slowly cooled to create precipitation, and samples of the precipitant are preferably obtained to ensure combination has been achieved and for quality control 26.

The precipitant now contains ajuga 12 embedded in cyclodextrin 14 together with the organic, food grade thickening excipient 16. The precipitant is next introduced to a precipitate filtration system 28, which separates out any remaining solids from the mixture, leaving only the precipitant. From the precipitate filtration system 28, the precipitant is next pumped to a tank for storage 30, and further samples are preferably obtained from the tank 30 and tested.

Following the second set of sampling, the precipitant is pumped into the fluid bed drying system 32. The fluid bed drying system is preferably equipped with a flow indicator 34 and a temperature indicator 36 of its own. The flow indicator 34 keeps track of the amount of precipitant being introduced to the fluid bed drying system 32 to ensure it is not over-supplied, while the temperature indicator 36 tracks the temperature of the precipitant to ensure it maintains a temperature range necessary for the fluidized bed drying process. The temperature indicator 36 also preferably verifies the temperature of the hot air being introduced into the fluid bed drying system 32.

A bag filter 38 is provided, in preferable embodiments as depicted in FIG. 1, to assist in releasing exhaust fumes 40 from the fluid bed drying system 32 when the drying process is in progress. And hot air 42 is continually introduced to the fluid bed drying system 32, as needed, during the drying process. In preferable embodiments, the hot air is heated to between 50 and 70 degrees Celsius before being introduced into the fluid bed drying system 32.

Once the drying process is completed, the dried granules of precipitant 44 are removed, and a third round of sampling occurs for quality control. At this stage the quality control process 46 ensures that the moisture levels in the dried granules of precipitant 44 are between three and five percent (3-5%). The quality control process 46 will either accept 48 or reject 50 the samples if they fall or do not fall in the required range.

After the quality control process 46 and acceptance 48 of the product output, the dried granules of precipitant 44 are transferred into polylined tote bins 52, which preferably undergo labeling 54 for tracking and compliance with good manufacturing practices and requirements. The dried granules of precipitant 44 are next introduced to filtration 56 based on granule size, preferably using a 16-20 mesh, though other filtration practices are also possible, as those of skill in the art will understand.

Any oversized 58 dried granules of precipitant 44 are milled 60 and reintroduced to the filtration 56. The filtered granules 62 are next introduced to a rotary mixer 64 for final processing before packaging into final product form 66 and prepared for shipment 68. As noted, sampling and testing is preferably undertaken throughout the process for quality control and to adhere to good manufacturing practices and requirements.

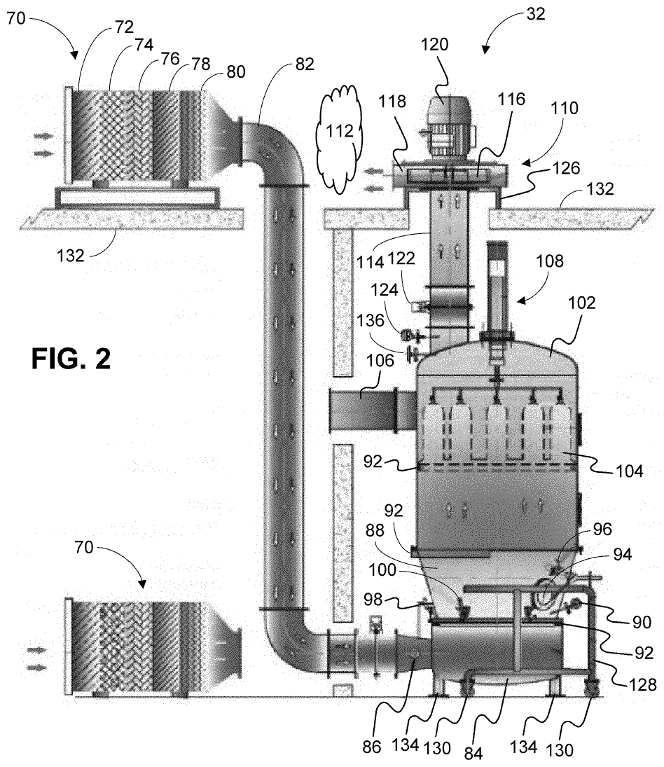

Referring now to FIG. 2, an exemplary embodiment of a fluid bed drying system 32 is depicted. The fluid bed drying system 32 preferably includes an air inlet filtration system 70 with a number of filters and other processing features to ensure the air introduced into the fluid bed drying system 32 is hot, dry, and free of contaminants. The filters and processing features include, in preferable embodiments, one or more of a course filter 72, a fine filter 74, a dehumidifier 76, a steam radiator and/or heater 78, and a hepa filter 80. The course filter 72 and fine filter 74 operate to remove contaminants from the intake air. The dehumidifier 76 removes moisture to ensure dry heat is used in the fluid bed drying system 32. The steam radiator and/or heater 78 heat the air up, preferably to within the 50 to 70 degrees Celsius range. Finally, the hepa filter 80 removes any additional particles or contaminants not filtered previously.

Once the intake air has passed through the air inlet filtration system 70, it is funneled 82 into the fluid bed drying system's 32 planium chamber 84. Preferable embodiments also include an inlet temperature sensor 86 to confirm the intake air is in the required temperature range. Above the planium chamber 84 is the product container 88, which holds the precipitant. A product temperature sensor 90 may be included to determine the temperature of the precipitant within the product container 88. An inflatable gasket 92 may be provided between the planium chamber 84 and the product container 88, in some preferable embodiments.

Also preferably included in the walls of the product container 88 is one or more viewing ports 94. The viewing ports 94 are covered by glass to maintain a fully enclosed environment within the product container 88 but allowing visibility of the precipitant within. Preferable embodiments of the product container 88 also include a product sampling port 96, as depicted in FIG. 2, that allows a user to access the precipitant held within.

A bowl earthing 98 is also preferably provided in the side of the product container 88 to assist in discharging built up electrical energy from the drying process. And an SS clamp 100 may be provided for locking the bottom mesh of the product container 88.

Above the product container 88 preferably resides an expansion chamber 102. The expansion chamber 102 contains one or more filter bags 104, and an explosion port 106 is preferably provided on at least one side of the expansion chamber 102. Also within the expansion chamber 102 is another inflatable gasket 92, in preferable embodiments, which preferably separates the one or more filter bags 104 from the remainder of the expansion chamber 102. Another inflatable gasket 92 may be provided between the product container 88 and expansion chamber 102, in some preferable embodiments, as depicted in FIG. 2. One or more viewing ports 94 may also be provided in the sides of the expansion chamber 102.

Provided in the top of the expansion chamber 102 is a telescoping cylinder assembly 108, which can operate to shake and agitate the one or more filter bags 104 during the drying process. The telescoping cylinder assembly 108 is preferably electronically operated, although those of skill in the art will recognize other approaches to providing the ability to agitate the one or more filter bags 104.

An exhaust system and assembly 110 is preferably provided to manage exhaust 112 created within the fluid bed drying system 32 during the drying process. The exhaust system and assembly 110 is preferably provided in the top of the expansion chamber 102 and includes a chimney 114 and a blower fan 116 provided within a blower casing 118 to facilitate removal of the exhaust 112 from the fluid bed drying system 32. The blower fan 116 is preferably operated by one or more electric motors 120. The chimney 114 is preferably provided with a damper 122 with an actuator for adjusting the damper 122. Some preferable embodiments of the chimney 114 are also provided with an outlet temperature sensor 124, preferably located between the expansion chamber 102 and the damper 122 to provide the most accurate temperature measurements. Some preferable embodiments of the chimney are also provided with a sensor for solid flow monitoring 136.

In the preferable embodiment depicted in FIG. 2, the fluid bed drying system 32 is located near a wall of the structure 132 such that the explosion port 106 can extend through the wall of the structure 132 to reach outside or into another room, for example. Likewise, the air inlet filtration system 70 and/or the funnel 82 may be provided outdoors or in another room, as depicted. And at least portions of the exhaust system and assembly 110 may extend upwardly through the roof of the structure 132 to reach the outside or another room, facilitating release of exhaust 112.

In such preferable embodiments, features of the fluid bed drying system 32, such as the exhaust system and assembly 110, may be mounted to the walls/roof of the structure 132 using mounting features 126, as depicted in FIG. 2. Preferable embodiments of the fluid bed drying system 32 may also employ a trolley system 128 to facilitate positioning and orientation of the fluid bed drying system 32 or portions thereof. As depicted in FIG. 2, such trolley systems preferably employ lockable wheels 130 to roll the fluid bed drying system 32, or portions thereof, along the ground. One or more feet 134 may also be provided extending from the bottom of the planium chamber 84 to stabilize the fluid bed drying system 32 once it is positioned and at rest.

While the present invention has been described with reference to particular processes, methods, embodiments, and arrangements of parts, features, and the like, it is not limited thereby. Indeed, modifications and variations will be ascertainable to those of skill in the art, all of which are inferentially and inherently included in these teachings.

What is claimed is:

1. A nutritional supplement comprising a complex of an ecdysterone and cyclodextrin, the complex comprising a one-to-one ratio of the ecdysterone and the cyclodextrin.

2. The nutritional supplement complex of claim 1, wherein the ecdysterone is turkesterone.

3. The nutritional supplement complex of claim 2, wherein the turkesterone is derived from ajuga turkestanica extract standardized to 10% turkesterone.

4. The nutritional supplement complex of claim 3 further comprising a thickening excipient.

5. The nutritional supplement complex of claim 4, wherein the thickening excipient is an organic, food grade gum.

6. The nutritional supplement complex of claim 4, wherein the complex is manufactured using a fluid bed drying and complexing process.

7. The nutritional supplement complex of claim 6, wherein the fluid bed drying and complexing manufacturing process comprises the steps of:
mixing, using a mixer, the ajuga turkestanica extract, the cyclodextrin, the thickening excipient, and hot water;
heating the mixture of the ajuga turkestanica extract, the cyclodextrin, the thickening excipient, and hot water to a predetermined temperature and for a predetermined period of time;
cooling the mixture of the ajuga turkestanica extract, the cyclodextrin, the thickening excipient, and hot water to cause precipitation;
separating, using a filtration system, and collecting the precipitant resulting from the precipitation during cooling;
drying the precipitant using a fluid bed drying system, resulting in dried granules of precipitant;
testing the dried granules of precipitant for potency and moisture concentration, and accepting for further processing only those dried granules of precipitant with a moisture concentration between three and five percent;
milling the dried granules of precipitant until they pass a predetermined granule size filtration system; and
packaging the dried, filtered granules of precipitant into a final product for distribution.

8. The fluid bed drying and complexing manufacturing process of claim 7, wherein the predetermined granule size filtration system comprises a 16-20 mesh.

9. The fluid bed drying and complexing manufacturing process of claim 7, wherein the fluid bed drying system comprises:
an air inlet filtration system comprising one or more of a course filter, a fine filter, a dehumidifier, a heater, and a hepa filter;
a planium chamber for receiving air filtered through the air inlet filtration system;
a product container for containing the precipitant, the product container comprising a product sampling port providing access to the precipitant held within the product container;
an expansion chamber comprising an explosion port, a telescoping cylinder assembly, and an exhaust system, the expansion chamber containing one or more filter bags, the telescoping cylinder assembly operating to agitate the one or more filter bags within the expansion chamber, and the exhaust system comprising:
a chimney with a damper and a damper actuator to control the flow of fluid up the chimney;
a blower casing containing one or more blower fans operated by one or more electric motors; and
an exhaust spout releasing the exhaust into the ambient environment.

10. The fluid bed drying and complexing manufacturing process of claim 9, wherein:
the fluid bed drying system further comprises one or more inflatable gaskets located in one or more of the following: between the planium chamber and the product container, between the product container and the expansion chamber, and within the expansion chamber;
the planium chamber of the fluid bed drying system further comprises an air inlet temperature sensors;
the product container of the fluid bed drying system further comprises a product temperature sensor, a bowl earthing, a ss clamp for locking a bottom mesh of the product container, and one or more glass viewing ports;
the expansion chamber of the fluid bed drying system further comprises one or more glass viewing ports; and
the chimney of the exhaust system of the fluid bed drying system further comprises an outlet temperature sensor and a sensor for solid flow monitoring, each located on the chimney between the damper and the expansion chamber.

11. A process for manufacturing a turkesterone hydroxypropyl β-cyclic dextrin complex comprising the steps of:
obtaining ajuga turkestanica extract standardized to 10% turkesterone, cyclodextrin, and an organic, food grade thickening excipient;
mixing, using a mixer, the ajuga turkestanica extract, the cyclodextrin, and the organic, food grade thickening excipient with hot water, wherein the ajuga turkestanica extract and the cyclodextrin are added to the mixer using a one-to-one ratio;
heating the mixture of the ajuga turkestanica extract, the cyclodextrin, the thickening excipient, and water to a predetermined temperature and for a predetermined period of time;
cooling the mixture of the ajuga turkestanica extract, the cyclodextrin, the thickening excipient, and water to cause precipitation;
separating, using a filtration system, and collecting the precipitant resulting from the precipitation during cooling;
drying the precipitant using a fluid bed drying system, resulting in dried granules of precipitant;
testing the dried granules of precipitant for potency and moisture concentration, and accepting for further processing only those dried granules of precipitant with a moisture concentration between three and five percent;
milling the dried granules of precipitant until they pass a predetermined granule size filtration system; and
packaging the dried, filtered granules of precipitant into a final product for distribution.

12. The process for manufacturing a turkesterone hydroxypropyl β-cyclic dextrin complex of claim 10, wherein the predetermined granule size filtration system comprises a 16-20 mesh

13. The process for manufacturing a turkesterone hydroxypropyl β-cyclic dextrin complex of claim 10, further comprising the step of extracting samples for quality control and to ensure adherence with good manufacturing practices throughout the process.

14. The process for manufacturing a turkesterone hydroxypropyl β-cyclic dextrin complex of claim 10, further comprising the step of transferring the dried granules of precipitant with a moisture concentration between three and five percent into poly-lined tote bins, which undergo labeling for tracking and compliance with good manufacturing practices and requirements.

15. The process for manufacturing a turkesterone hydroxypropyl β-cyclic dextrin complex of claim 10, further comprising the step of adding the dried, filtered granules of precipitant to a rotary mixer for final processing before packaging the dried, filtered granules of precipitant into a final product for distribution.

16. The process for manufacturing a turkesterone hydroxypropyl β-cyclic dextrin complex of claim 10, wherein the mixer comprises a reaction progress monitoring system and a temperature indicator, the reaction progress monitoring system obtaining data about the mixture of the ajuga turkestanica extract, the cyclodextrin, the thickening excipient, and water from the mixer, and the temperature indicator providing the reaction progress monitoring system with temperature information about the mixture of the ajuga turkestanica extract, the cyclodextrin, the thickening excipient, and water in the mixer.

17. The process for manufacturing a turkesterone hydroxypropyl β-cyclic dextrin complex of claim 10, wherein the fluid bed drying system comprises a flow indicator and at least one temperature indicator, the flow indicator operating to prevent over-supply of precipitant into the fluid bed drying system, and the at least one temperature indicator monitoring the temperature of hot air introduced to fluid bed drying system, which should be between 50 and 70 degrees Celsius.

18. The process for manufacturing a turkesterone hydroxypropyl β-cyclic dextrin complex of claim 10, wherein the fluid bed drying system comprises:
an air inlet filtration system comprising one or more of a course filter, a fine filter, a dehumidifier, a heater, and a hepa filter;
a planium chamber for receiving air filtered through the air inlet filtration system;
a product container for containing the precipitant, the product container comprising a product sampling port providing access to the precipitant held within the product container;
an expansion chamber comprising an explosion port, a telescoping cylinder assembly, and an exhaust system, the expansion chamber containing one or more filter bags, the telescoping cylinder assembly operating to agitate the one or more filter bags within the expansion chamber, and the exhaust system comprising:
a chimney with a damper and a damper actuator to control the flow of fluid up the chimney;
a blower casing containing one or more blower fans operated by one or more electric motors; and
an exhaust spout releasing the exhaust into the ambient environment.

19. The process for manufacturing a turkesterone hydroxypropyl β-cyclic dextrin complex of claim 18, wherein:
the fluid bed drying system further comprises one or more inflatable gaskets located in one or more of the following: between the planium chamber and the product container, between the product container and the expansion chamber, and within the expansion chamber;
the planium chamber of the fluid bed drying system further comprises an air inlet temperature sensors;
the product container of the fluid bed drying system further comprises a product temperature sensor, a bowl earthing, a ss clamp for locking a bottom mesh of the product container, and one or more glass viewing ports;
the expansion chamber of the fluid bed drying system further comprises one or more glass viewing ports; and
the chimney of the exhaust system of the fluid bed drying system further comprises an outlet temperature sensor and a sensor for solid flow monitoring, each located on the chimney between the damper and the expansion chamber.
